(19) United States
(12) Patent Application Publication
(10) Pub. No.: US 2026/0295302 A1
(43) Pub. Date: Oct. 1, 2026

(54) RADIOTHERAPY SYSTEMS HAVING ONE OR MORE EVENT CAMERAS

(71) Applicant: Siemens Healthineers International AG, Steinhausen (CH)

(72) Inventors: Pascal PAYSAN, Basel (CH); Stephen THOMPSON, Marina, CA (US); Roland MEIER, Herzogenbuchsee (CH); Wendy SMITH, Calgary (CA); Michael FOLKERTS, Costa Mesa, CA (US)

(73) Assignee: Siemens Healthineers International AG, Steinhausen (CH)

(21) Appl. No.: 19/095,829

(22) Filed: Mar. 31, 2025

(51) Int. Cl. A61N 5/10

(52) U.S. Cl. CPC A61N 5/1049; A61N 2005/105; A61N 2005/1059

(57) ABSTRACT

At least one example embodiment provides a system comprising a radiotherapy machine; at least one event camera; and processing circuitry configured to operate at least one of the radiotherapy machine or the at least one event camera.

### TECHNICAL FIELD

One or more example embodiments relate to radiotherapy systems having one or more event cameras.

### BACKGROUND

Radiation therapy involves medical procedures that selectively expose certain areas of a human body, such as cancerous tumors, to doses of radiation. The radiation therapy irradiates the targeted biological tissue such that undesirable tissue is destroyed. Radiation has also been-used to obtain images of tissue for diagnostic or treatment purposes.

Optical devices such as stereoscopic (3D) cameras, 2D cameras, time-of-flight (ToF) cameras, LiDAR, or structured light cameras are often used in radiation systems to aid patient setup, verify patient identification, monitor treatment, or provide guidance such as in surface guided radiotherapy (SGRT).

### SUMMARY

The scope of protection sought for various example embodiments is set out by the independent claims. The example embodiments and/or features, if any, described in this specification that do not fall under the scope of the independent claims are to be interpreted as examples useful for understanding various embodiments.

According to one or more example embodiments, a system includes a radiotherapy machine; at least one event camera; and processing circuitry configured to operate at least one of the radiotherapy machine or the at least one event camera.

According to one or more example embodiments, a system includes a radiotherapy machine; at least one event camera; and means for operating at least one of the radiotherapy machine or the at least one event camera.

### BRIEF DESCRIPTION OF THE DRAWINGS

Example embodiments will become more fully understood from the detailed description given herein below and the accompanying drawings, wherein like elements are represented by like reference numerals, which are given by way of illustration only and thus are not limiting of this disclosure.

FIGS. 1A-1B illustrate a system according to at least one example embodiment;

FIG. 1C illustrates a system according to at least one example embodiment;

FIG. 2 illustrates an example embodiment of a medical system including at least one event camera;

FIG. 3 illustrates a motion measuring apparatus according to at least one example embodiment;

FIG. 4 illustrates a flowchart of a method for a radiotherapy system, according to one or more embodiments; and

FIG. 5 illustrates a diagram of a control system with which embodiments may be implemented.

It should be noted that these figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment and should not be interpreted as defining or limiting the range of values or properties encompassed by example embodiments. The use of similar or identical reference numbers in the various drawings is intended to indicate the presence of a similar or identical element or feature.

### DETAILED DESCRIPTION

Various example embodiments will now be described more fully with reference to the accompanying drawings in which some example embodiments are shown.

Detailed illustrative embodiments are disclosed herein. However, specific structural and functional details disclosed herein are merely representative for purposes of describing example embodiments. The example embodiments may, however, be embodied in many alternate forms and should not be construed as limited to only the embodiments set forth herein.

It should be understood that there is no intent to limit example embodiments to the particular forms disclosed. On the contrary, example embodiments are to cover all modifications, equivalents, and alternatives falling within the scope of this disclosure. Like numbers refer to like elements throughout the description of the figures.

While one or more example embodiments may be described from the perspective of a particular device, it should be understood that one or more example embodiments discussed herein may be performed by the one or more processors (or processing circuitry) at the applicable device. For example, according to one or more example embodiments, at least one memory may include or store computer program code, and the at least one memory and the computer program code may be configured to, with at least one processor, cause a device or system to perform the operations discussed herein.

As discussed herein the terminology “one or more” and “at least one” may be used interchangeably.

It will be appreciated that a number of example embodiments may be used in combination.

A major challenge in Surface-Guided Radiation Therapy (SGRT) is the use of camera-based acquisition systems. A camera-based acquisition system may use a combination of high spatial resolution, low latency and large Field-of-View (FoV) to cover possible use cases. Latency limitations are at least partially caused by the limited framerates of affordable conventional cameras and the desirability for continuous, fast processing of large data volumes. Often, real-time processing for large data volumes is limited.

In large FoVs, one limiting factor apart from a depth of field is a dynamic range of conventional camera-based systems resulting in constraints for lighting and surface properties. This introduces uncertainties in applications of the camera system output, such as treatment gating or 4D imaging. Moreover, the useability of such systems for patient-machine collisions is limited.

To address these limitations the framerate of conventional cameras may be increased and/or different projected light patterns may be applied, often combining with different exposure times for different scenes, and adding computing power (e.g., GPU processing).

However, an increased framerate causes under-exposure or requires intense lighting (limitations in the treatment room) and higher data rates that need to be processed (e.g., creating a bottleneck or requiring expensive hardware). The limited dynamic range of conventional cameras imposes constraints on lighting and potentially causes difficulties in handling dark skin and overexposures due to spotlights and bright surfaces. The limited frame rate restricts the application to static mounting, i.e., forces a room-fixed mounting. While a couch-mount camera avoids the interference and occlusion by a moving gantry and/or detectors blocking the line of sight, the couch-mount introduces vibrations that requires higher frame rates and limits camera angles.

Example embodiments provide a radiation treatment system that includes one or more event cameras. The one or more event cameras may be used to monitor a patient or in combination with an existing frame-based camera. The one or more event cameras may be used to monitor a patient support (e.g., table, fixation) for SGRT as well as for a collision avoidance system. The use of one or more event cameras improves the latency of these systems relative to the use of conventional cameras in these systems. In some example embodiments, the one or more event cameras are realized with a passive stereo (or multi-view) camera system and/or as an active system using structured light projectors (or a raster scanning laser point projector).

Event cameras are not tied to the inverse relation between framerate and spatial resolution. Low latencies can be achieved with a fraction of the data volume conventional high-speed cameras typically provide and can thus still provide the sub-mm spatial resolution for certain radiotherapies such as Stereotactic Radiosurgery (SRS). Overexposures are reduced or eliminated using event cameras, and underexposures are limited by the noise level of the background. Using an event camera reduces or eliminates spotlight, overexposed scenes and removes the need to make a distinction between light and dark skin patients.

In some example embodiments, event cameras only record motion. The event cameras may be used for patient setup as well as after or at least one event camera may be used in conjunction with an optical or X-ray system for patient setup. For example, after a patient is set up using an optical or X-ray system, one or more event cameras are used to monitor deviations, i.e., motions relative to the setup using the optical or X-ray system. The event camera may detect voluntary (such as fidgeting, positional adjustments) and involuntary (such as breathing, coughing, tensing or relaxation) patient motion. Moreover, one or more event cameras may be used to avoid collisions by monitoring movement of the patient, accessories, and a linear accelerator system, including the gantry, couch, imaging arms.

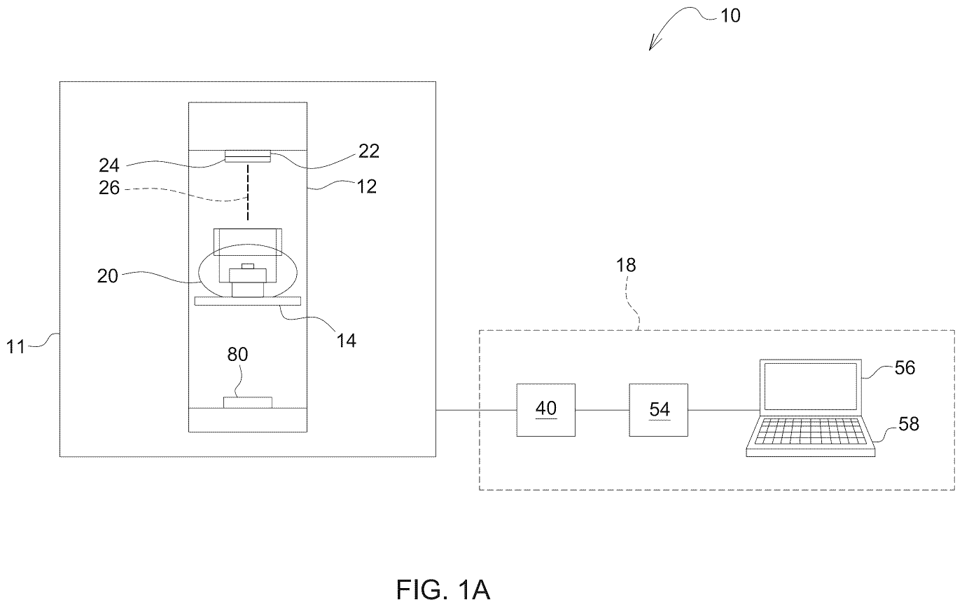

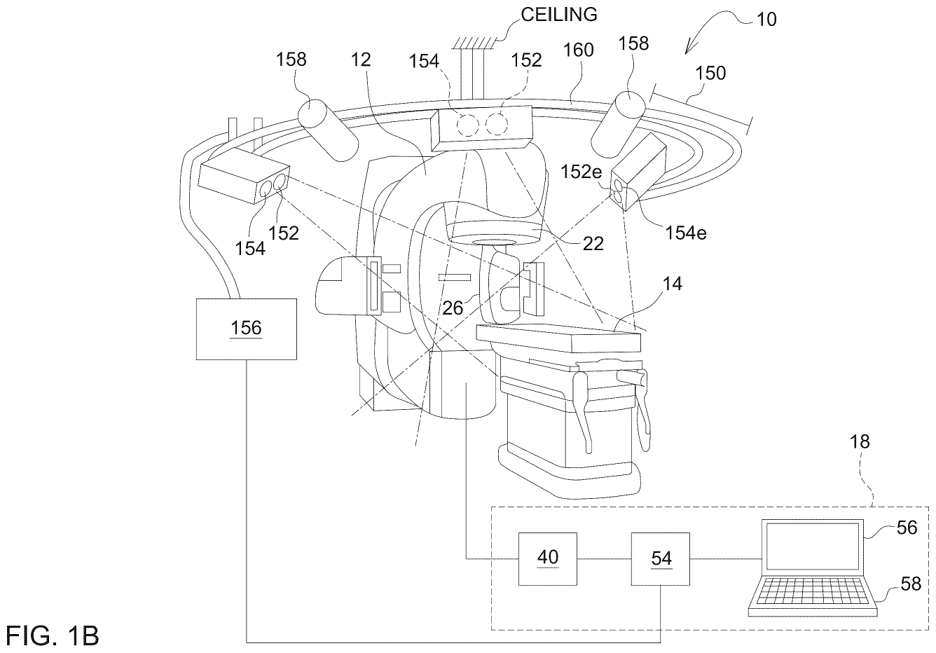

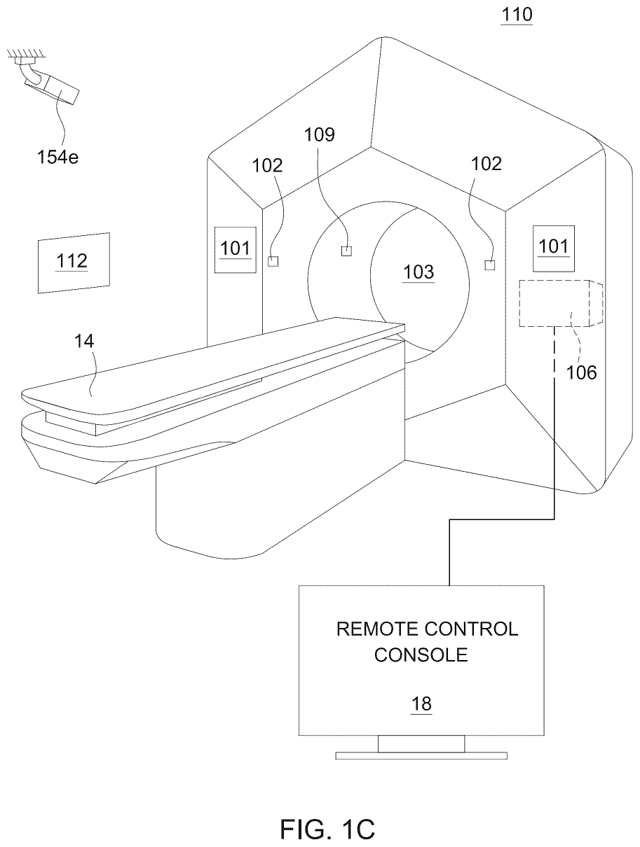

FIGS. 1A-1B illustrate a system according to at least one example embodiment.

FIG. 1A illustrates a medical system 10. In the illustrated embodiment, the medical system 10 is a radiation treatment system (radiotherapy system), and it includes a medical device 11 (radiotherapy machine) and a patient support 14 for supporting a patient 20. The medical device 11 includes an arm gantry 12 (in this concrete embodiment a C-arm gantry) and a control system 18 for controlling an operation of the gantry 12 and delivery of radiation. The medical device 11 also includes a radiation source 22 (e.g., a linear accelerator (LINAC)) that projects a beam 26 of radiation towards the patient 20 while the patient 20 is supported on support 14, and a collimator 24 for changing a cross sectional shape of the beam 26. The radiation source 22 may be configured to generate a cone beam, a fan beam, or other types of radiation beams in different embodiments. Also, in other embodiments, the source 22 may be configured to generate proton beam as a form of radiation for treatment purpose. Also, in other embodiments, the system 10 may have other form and/or configuration. For example, in other embodiments, instead of an arm gantry 12, the medical device 11 may have a ring gantry or a robotic arm.

In the illustrated embodiments, the radiation source 22 is a treatment radiation source for providing treatment energy. In other embodiments, in addition to being a treatment radiation source, the radiation source 22 can also be a diagnostic radiation source for providing diagnostic energy for imaging purpose. In such cases, the system 10 will include an imager 80, such as the imager 80, located at an operative position relative to the source 22 (e.g., under the support 14). In further embodiments, the radiation source 22 may be a treatment radiation source for providing treatment energy, wherein the treatment energy may also be used to obtain images. In such cases, in order to obtain imaging using treatment energies, the imager 80 is configured to generate images in response to radiation having treatment energies. In some embodiments, the treatment energy is generally those energies of 160 kilo-electron-volts (keV) or greater, and more typically 1 mega-electron-volts (MeV) or greater, and diagnostic energy is generally those energies below the high energy range, and more typically below 160 keV. In other embodiments, the treatment energy and the diagnostic energy can have other energy levels and refer to energies that are used for treatment and diagnostic purposes, respectively. In some embodiments, the radiation source 22 is able to provide X-ray radiation at a plurality of photon energy levels within a range anywhere between approximately 10 keV and approximately 20 MeV. In further embodiments, the radiation source 22 can be a diagnostic radiation source. In such cases, the system 10 may be a diagnostic system with one or more moving parts. In the illustrated embodiments, the radiation source 22 is carried by the arm gantry 12. Alternatively, the radiation source 22 may be located within a bore (e.g., coupled to a ring gantry).

In further embodiments, one or more additional pairs of a diagnostic radiation source and an imager at a suitable operative position complement the treatment beam. Diagnostic radiation beam lines are typically at a certain angle with the treatment beam (e.g. one pair of a diagnostic source and a kV detector for energies below 160 keV are perpendicular to the treatment beam, intersecting the treatment beam at an isocenter).

Thus, the event camera(s) may be used for gating not just the treatment beam (e.g., turning on and off) but also to gate both the treatment and imaging systems depending on use case scenario. The treatment and imaging beams may be gated to prevent irradiation when the patient is out of position through an irregular motion, such as coughing, breath holds or tensing of muscles, or though repetitive motion, like respiration. For repetitive motion, such as breathing, this may be based on phase or amplitude. Alternatively, the time information from repetitive motion as assessed by the event cameras may be recorded and used for reconstruction of images (e.g. 4DCT) or dose delivered. Additionally, the event camera(s) can be used to track internal targets (e.g., a target volume) based on the event data captured from the event camera(s) to determine whether the internal target is outside of a target/reference position. Thus, the treatment and imaging beams may be gated to prevent irradiation when the internal target is outside of a target position. In some embodiments, the tracking of internal targets may be accomplished by use of a motion model and event data capture from the event camera(s).

In the illustrated embodiments, the control system 18 includes a processing circuitry 54, such as a processor, coupled to an input/output device 40. The control system 18 may also include a monitor 56 for displaying data and an input device 58, such as a keyboard or a mouse, for inputting data. The operation of the radiation source 22 and the gantry 12 are controlled by the processing circuitry 54, which provides power and timing signals to the radiation source 22, and controls a rotational speed and position of the gantry 12, based on signals received from the processing circuitry 54. In some cases, the processing circuitry 54 may also control the radiation source 22 and the position of the patient support 14. In addition, in some cases, the processing circuitry 54 may be configured to control the beam 26 (e.g., beam hold for gating). Furthermore, the processing circuitry 54 may be configured to control an imaging process (e.g., triggering of imaging) using the treatment radiation source 22 or one or more dedicated imaging radiation sources. Although the input/output device 40 is shown as a separate component from the gantry 12 and the processing circuitry 54, in alternative embodiments, the input/output device 40 can be a part of the processing circuitry 54.

In some embodiments, the medical system 10 may be a treatment system configured to deliver treatment radiation beam towards the patient 20 at different gantry angles. During a treatment procedure, the source 22 rotates around the patient 20 and delivers treatment radiation beam from different gantry angles towards the patient 20. While the source 22 is at different gantry angles, the collimator 24 is operated to change the shape of the beam to correspond with a shape of the target tissue structure. For example, the collimator 24 may be operated so that the shape of the beam is similar to a cross sectional shape of the target tissue structure. In another example, the collimator 24 may be operated so that different portions of the target tissue structure receive different amount of radiation (as in an intensity-modulated radiation therapy (IMRT) procedure).

In other embodiments, the medical system 10 may be an imaging system configured to deliver imaging radiation beam towards the patient 20 for imaging the patient 20. In other embodiments, the imaging system may be any other imaging system as known in the art, such as but not limited to, MRI, PET, and SPECT.

As shown in FIG. 1B, the system 10 also includes an optical system 150. The optical system 150 may include a light source 152, multiple cameras 154 (e.g., stereo cameras), and a processing unit 156 in communication with the cameras 154. In the illustrated example, the light source 152 is configured to provide structured light and/or non-structured light. Also, as shown in the figure, the optical system 150 has three cameras 154. In some embodiments, the processing unit 156 can be a distributed control unit with processing happening in each of the pods, where a pod constitutes of a light source 152 and one or multiple cameras 154.

At least one of the three cameras 154 is an event camera 154e and the remaining cameras may be frame-based cameras. In some example embodiments, the event camera is used for 3D surface tracking for the patient. Additionally, or alternatively, the event camera is used for at least one of collision avoidance, patient positioning, patient monitoring, motion management or respiratory gating. In some example embodiments, the event camera 154e tracks a marker block or one or more markers affixed to or resting on a patient. Additionally, or alternatively, the event camera 154 may track tools.

In other embodiments, the optical system 150 has more than one event camera, fewer than three cameras 154 (e.g., one camera 154 or two cameras), or more than three cameras 154. Also, in other embodiments, the optical system 150 may include multiple light sources 152.

The event camera 154e may have a laser source 152e to perform laser scanning and track motion.

The optical system 150 may also optionally include a frame 160 to which the cameras 154 and the light source 152 may be mounted. The frame 160 may be mounted to a ceiling and/or a wall of a room in which the medical system 10 is located. Alternatively, the frame 160 may be mounted to the medical system 10. The cameras 154 may be moveably mounted to the frame 160. In one implementation, each of the cameras 154 may be rotatably mounted to the frame 160 (e.g., via a ball joint) so that the camera 154 is rotatable about one or more axes with respect to the frame 160. Similarly, the light source 152 may be moveably mounted to the frame 160. For example, the light source 152 may be rotatably mounted to the frame 160 (e.g., via a ball joint) so that the light source 152 is rotatable about one or more axes with respect to the frame 160. In other embodiments, instead of ball joints, the cameras 154 and the light source 152 may be moveably mounted to the frame 160 using other connectors, such as arms, so that the cameras 154 and the light source 152 are moveable with respect to the frame 160. In other embodiments, the one or more of the cameras 154 and/or the light source 152 may be mounted directly to the medical system 10 or a room.

As shown in FIG. 1B, the optical system 150 may also include a plurality of time-of-flight (TOF) cameras 158. Each TOF camera 158 is configured to provide depth image(s). A depth image has pixel values representing a distance between a reference point and a surface point detected. In some embodiments, each TOF camera 158 may be an infrared camera. During use, images from the cameras 154 and the TOF cameras 158 are processed by the processing unit 156 to obtain and monitor surface contours of the patient before and during treatment for the purpose of patient setup (absolute positioning and/or relative positioning), patient monitoring during treatment (e.g., monitoring absolute position and/or relative position), tool surveillance, prevention of patient-machine collisions, or a combination of the foregoing. Patient monitoring may include: (1) ensuring that the patient does not leave its setup position, and/or (2) recording a periodic patient motion due to breathing, and controlling a machine accordingly (e.g., beam hold, multi-leaf collimator tracking, tracking of patient support, etc.).

In some cases, the TOF cameras 158 may help increase a field of view, and may observe blind spots not captured by the camera(s) 154.

In other embodiments, the optical system 150 may not include any TOF cameras 158.

In some embodiments, the optical system 150 may include multiple pods, wherein each pod may have one or more light sources 152, one or more cameras 154 (e.g., two cameras 154), and one or more TOF cameras 158. For example, there may be a first pod having one or more light sources 152 and two cameras 154, and a second pod having one or more light source 152 and two cameras 154.

The at least one event camera 154e may be used to detect markers with known geometric properties to obtain geometric 3D information.

In some embodiments, the pod(s) may be mounted to a frame of the optical system 150. In other embodiments, the pod(s) may be mounted to a different frame than that of the optical system 150. Also, in further embodiments, the pod(s) may be configured to be mounted to the medical system 10, e.g., to the gantry 12 (e.g., to the radiation source 22 or embedded in the arm of the gantry) and/or to the patient support 14. In some cases, the pod(s) may be mounted to deployable arms that are coupled to the medical system 10. In other embodiments, the pod(s) may be mounted to a room (e.g., to a wall, a ceiling, a floor, etc.).

The optical system 150 may be configured to provide patient setup, patient monitoring, device monitoring, respiratory motion monitoring, patient-machine collision prevention, or any combination of the foregoing. Thus, in some cases, the same optical system 150 may provide multiple purposes. In some embodiments, different clinical use cases mentioned above may be performed simultaneously. In one implementation, the sequence of real-time input images from the frame-based camera(s) 154, the at least one event camera 154e and from the TOF camera(s) 158 may be processed by the processing unit 156 to patient monitoring and/or device monitoring.

In one method of use, the light source 152 of the optical system 150 may be used to provide structured light. The structured light may be projected onto an object, such as a patient, for patient setup. As used in this specification, when light is described as being projected onto a patient, it is intended to cover the scenario in which the light is projected directly onto the patient (i.e., onto the skin of the patient), as well as the scenario in which the light is projected onto an object worn or coupled to the patient (e.g., onto a garment worn by the patient, a blanket covering the patient, a sticker on the patient, patient immobilization device, etc.). The cameras 154 sense the structured light as projected on the patient, and generate images of the projected structured light. Additionally, the laser source 152e is controlled to scan (e.g., a raster scan) the patient. The changes in illumination are detected by the event camera 154e, which causes events to be generated by the event camera 154e.

The processing unit 156 is configured to process the images from the cameras 154, process the events from the event camera 154e, and determine a position (e.g., location and/or orientation) of the patient based on the processed images and events. Once the position of the patient is determined, the processing unit 156 may determine which direction to move the patient, and how much to move the patient, based on a desired position of the patient to be achieved.

In at least one example embodiment, the light source 152 projects a laser onto the patient and the event camera captures event data which corresponds to movement of the laser on the patient. The processing unit 156 is configured to obtain a measured surface of the patent based on the captured event data.

In at least one example embodiment, the event camera 154e and laser source 152e may be mounted to the gantry 12. The event camera would allow confirmation of whether a predicted scene matches an aligned scene. More specifically, the gantry mounted optical system (i.e., the camera 154 e and the light source 152 e) detects a 3D geometry of the scenery. By applying (inverting) a known geometric orientation of the gantry 12, the processing unit 156 can obtain the 3D geometry of the room including the patient. The processing unit 156 can then compare the obtained 3D geometry with an expected scene (e.g., is the patient at the right position, did a person move in the way of the machine, or is some accessory in the way).

In some cases, the system (camera, light source) delivers a point cloud (calculated according to stereo vision principles). The 3D points represent surface points that can be geometrically analyzed. The patient may then be re-positioned based on the point cloud.

In some embodiments, the light source 152e provides structured light (e.g., raster laser scan) and directs it onto an object (e.g., a patient), and the reflected light from the object is detected by the event camera 154e (e.g., as an event), which is offset from the light source 152e (e.g., an active system). The geometry of the light source 152e and the event camera 154e is known. Accordingly, the processing unit 156 can use triangulation to calculate the distance of to the surface of the object.

In some cases, the light source 152e and the event camera 154e may be implemented as one pod, and there may be additional pod(s), wherein each pod has a light source and an event camera 154e. The processing unit 156 may be configured to combine the determined surface from one pod to other surfaces determined from other pod(s) at other locations in order to map out the surface of the object, thereby forming a larger surface of the object. In some example embodiments, the pods (with each pod includes a light source and event camera) may be placed around the object, which avoids/reduces occlusion by the radiation therapy machine and/or occlusion due to the curvature of the object. In addition or alternatively, the pods (with each pod includes a light source and event camera) may be placed along the object (e.g., in front and behind a bore 103 (in FIG. 1C). The processing unit 156 may also calculate the distance of a reference surface to a measured surface to detect a possible offset.

In some embodiments, the surface may be determined using two event cameras 154e instead of a using a light source (e.g., a passive system). In such cases, the event cameras 154e detect events based on surface reflection of the object that are caused due to motion of the object. The processing unit 156 then utilizes an algorithm to find the corresponding pixel in both event cameras. Knowing the camera pixel for this surface point and the event cameras configuration (e.g., position and/or orientation), the angle of the ray towards this object point can be determined by the processing unit 156 for each camera. As the distance between both event cameras 154e is known, triangulation technique may then be used by the processing unit 156 to calculate the distance to this surface point (also known as “distance of surface”). In some embodiments, such distance to the surface point may be measured from the camera pod. The above process may be repeated for all object points to thereby create a surface of interest in a known coordinate system. Similar methods can be used to estimate the amplitude and direction of motion and relative distances between objects.

The optical system 150 may be configured to provide patient setup, patient monitoring, device monitoring, respiratory motion control, patient-machine collision prevention, or any combination of the foregoing. Information from the at least one event camera 154e can be used to gate or steer the treatment beam, trigger and coordinate imaging sequences, change or adapt the motion systems of the medical system, and make decision regarding the course of the treatment or diagnostic imaging. Also, in some embodiments, by combining external surface information of the patient (provided by the event camera 154e) with x-ray imaging of the internal anatomy, highly integrated and automated treatment workflows may be achieved.

In a surface-based monitoring workflow, a user manually defines a region-of-interest (ROI) on a 3D surface image of a patient. The ROI is used by a treatment system (e.g., radiotherapy system) to measure motion such as respiratory motion.

FIG. 1C is a perspective view of a system 110, according to other various embodiments. In some embodiments, treatment delivery system 1100 includes an imaging system configured to image patient anatomy using X-ray imaging techniques. For example, in some embodiments, treatment delivery system 200 is configured to provide stereotactic radiosurgery and precision radiotherapy for lesions, tumors, and conditions anywhere in the body where radiation treatment is indicated. As such, treatment delivery system 1100 can include one or more of a linear accelerator (LINAC) that generates a megavolt (MV) treatment beam of high energy X-rays, one or more kilovolt (kV) X-ray sources, one or more X-ray imagers, and, in some embodiments, an MV electronic portal imaging device (EPID). By way of example, radiation therapy system 1100 is described herein configured with a circular gantry.

In some embodiments, treatment delivery system 1100 is capable of kV imaging of a target volume immediately prior to or during application of an MV treatment beam, so that an IGRT and/or an intensity-modulated radiation therapy (IMRT) process can be performed using X-ray imaging. Treatment delivery system 110 may include one or more touchscreens 201, couch motion controls 102, a bore 103, a base positioning assembly 105, the patient support 14, and an image acquisition and treatment control computer 106, all of which are disposed within a treatment room. Treatment delivery system 110 further includes the remote control console 18, which is disposed outside the treatment room and enables treatment delivery and patient monitoring from a remote location. A base positioning assembly is configured to the patient support 14 with respect to the bore 103, and motion controls 102 include input devices, such as button and/or switches, that enable a user to operate base positioning assembly to position the patient support 14 to a predetermined location with respect to the bore 103.

Patient-monitoring sensors 109 and the event camera 154e can be incorporated into a patient position-monitoring system or surface guidance system that can generate a map of the surface of a patient positioned on the patient support 14. In some example embodiments, event cameras may be used instead of all or some patient-monitoring sensors 109. Thus, at least one event camera 154e may be on a surface that defines the bore 103. In operation, the surface map and/or indicators showing offsets between a current patient position and a planned patient position are displayed on an output device, such as a display screen located proximate the patient support 14.

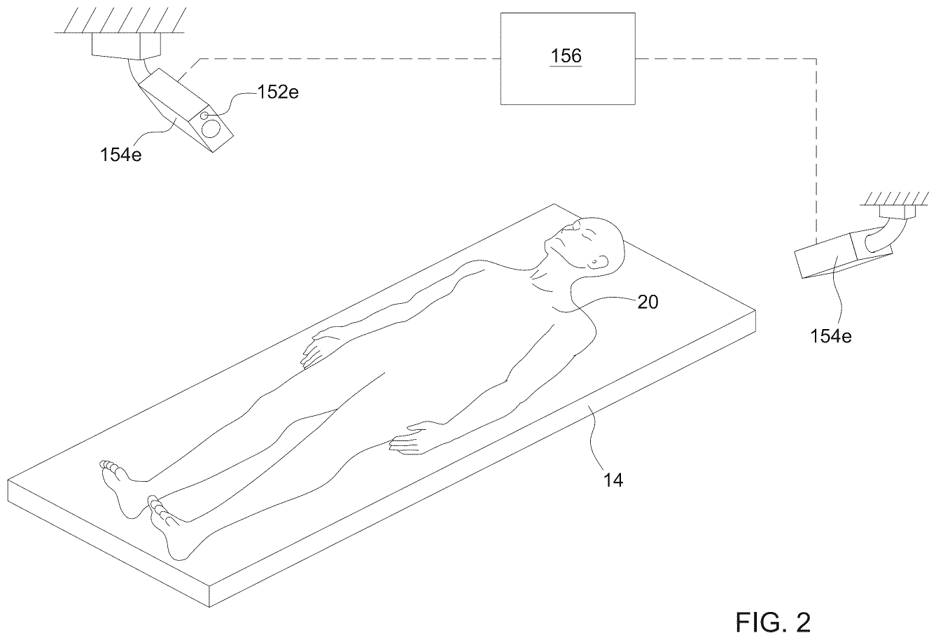

FIG. 2 illustrates an example embodiment of a medical system including two event cameras to obtain a 3D image from both sides of the patient to obtain a 3D model of the patient. In the example where the cameras are event cameras and the number of event cameras 154e is two, a surface model of the patient may be formed by combining event data from the different event cameras 154e. The two event cameras 154e may be positioned to view opposite sides of a patient 20. In particular, there is a first event camera 154e positioned to view the patient 20 from his/her right side, and a second event camera 154e positioned to view the patient 20 from his/her left side. In another example, there may be a first event camera 154e positioned to view the patient 20 from above his/her head towards a direction of the feet, and a second event camera 154e positioned to view the patient 20 from below his/her feet towards a direction of the heard. In some embodiments, the processing unit 156 may be configured to receive first event data (e.g., with first depth information) from the first event camera 154e, and to receive second event data (e.g., with second depth information from the second stereo camera 154e. Because the relative position between the two event cameras 154e is known, the processing unit 156 may use this positional information to perform coordinate transformation so that the surfaces of the patient 20 from the two images may be combined to form a 3D surface image of the patient.

Similarly, the processing unit 156 and/or processing circuitry 54 may determine a transformation between a frame of reference of the event camera and a frame of reference of the radiation source (e.g., LINAC).

In some embodiments, the processing unit 156 may be configured to determine motion of a surface from at least one of the event cameras 154e, or a merged multi-camera view based on expected and/or detected protected surface location and dimension. If the system includes multiple event cameras 154e, the multiple event cameras may be calibrated to align the cameras to produce a merged view.

In some embodiments, the processing unit 156 may generate the motion values and/or images as described in Muglikar, et al., ESL: Event-based Structured Light, 2021 Int. Conf. 3D Vis. (3DV) 00, 1165-1174 (2021), the entire contents of which are incorporated by reference.

In other example embodiments, a single event camera 154e and a single light source (e.g., laser 152e) may be used. Moreover, the single event camera 154e and a single light source may be separated (e.g., one of the event cameras 154e is replaced with just the light source and the other event camera 154e does not include a light source). The processing unit 156 may then perform a 3D estimation using epipolar geometry. The light source (e.g., laser) may point at a known geometry into the scene and the reflection on the surface may be detected by the single event camera 154 e. The relative 3D position with respect to the views can then be calculated using triangulation.

The two event cameras 154e may also be used for collision avoidance, repositioning of the patient, fixation device positioning, calibration phantom localization and surveillance of the room in which the system 10 is located.

The processing unit 156 is configured to process signals (event data) transmitted from the event cameras 154e, and to determine whether there is a possible collision between the patient and an object based on the signals.

In the illustrated embodiments, the processing unit 156 is configured for determining a first model based at least in part on the image, at least a part of the first model representing a surface of the patient. The processing unit 140 is also configured for determining a second model, the second model representing a first component of the medical system. It should be noted that as used in this specification, the term “component” may refer to any portion of a medical system, which portion may be a device or a part of a device. For example, with respect to the patient support 14, the component may be the patient support 14 itself, an individual part (such as a plate forming the support surface of the patient support 14), or a portion of the individual part of the patient support 14, such as a surface that is a subset of all of the surfaces of an individual part of the patient support 14. In another embodiment, the system 10 can for example also include a deployable imager and a diagnostic x-ray source, which are then modeled as part of the machine model, here denoted as second model representing a second component of the medical system.

Also, the processing unit 156 is configured to execute a collision prediction mechanism configured for virtually moving the first model, the second model, or both, to simulate a movement of the first component of the medical system to determine whether there is a possible collision between the first component and the patient.

In other example embodiments, the first model and the second model may represent surfaces of different components of the system 10. The processing unit 156 is configured to execute a collision prediction mechanism configured for virtually moving the first model, the second model, or both, to simulate a movement of the first component of the medical system to determine whether there is a possible collision between two components such as the gantry 12 and the patient support 14.

Also, the collision prediction mechanism may be configured for generating an output to indicate the possible collision or an absence of the possible collision. The collision prediction mechanism may be implemented as a module in the processing unit 156 or as a separate processing unit.

Also, in some embodiments, a safety margin may be provided for the patient surface that is desired to be protected. In such cases, the surface of the patient may be a protected surface that is away from the actual patient's surface. For example, the protected surface may be defined as a surface that is 5 cm (or any of other values) away from, and surrounding, the actual patient's surface. Thus, the “surface” of the patient may refer to the actual surface of the patient 20, a surface of object(s) connected to or extending from the patient 20, or a surface (e.g., a virtual surface that is the protected surface) that is away from and surrounding the actual patient's surface.

The images provided by the event cameras 154e may also include surface contours of other objects in the treatment room. For example, the images may include surface information regarding the patient support 14, the gantry 12, the collimator 24, the imager 80 (which may be a portal imager), and kV imager (e.g., which may include a deployable imager and a diagnostic x-ray source), which indicate the surface contours of these components.

In at least one example embodiment, the processing unit 156 processes the images to identify surfaces that are desired to be protected (e.g., to avoid pre-programmed machine motions causing patient-machine collisions, or during a treatment, observed patient motion not leading to a mistreatment or interfering with the expected machine motion). In some cases, this may be performed automatically.

In other cases, a user may provide input to the processing unit 156 to identify the different surfaces that are desired to be protected during the treatment procedure. In one implementation, each event camera 154e may also have optical imaging capability, and may provide an optical image for display on a screen. The user may view the optical image together with the image generate from the event data, in a side-by-side configuration or an overlay configuration, and may then identify different surfaces.

After different surfaces that are desired to be protected during the treatment procedure are identified, the surfaces may then be processed by the processing unit 156 to determine different models representing the different objects having the identified surfaces. In some cases, the surfaces themselves may be the models representing the different respective objects involved in the treatment procedure. In some embodiments, the models of the various objects may be stored in a non-transitory medium for later use. Also, in some embodiments, the models of the various objects may be displayed on a screen for presentation to a user.

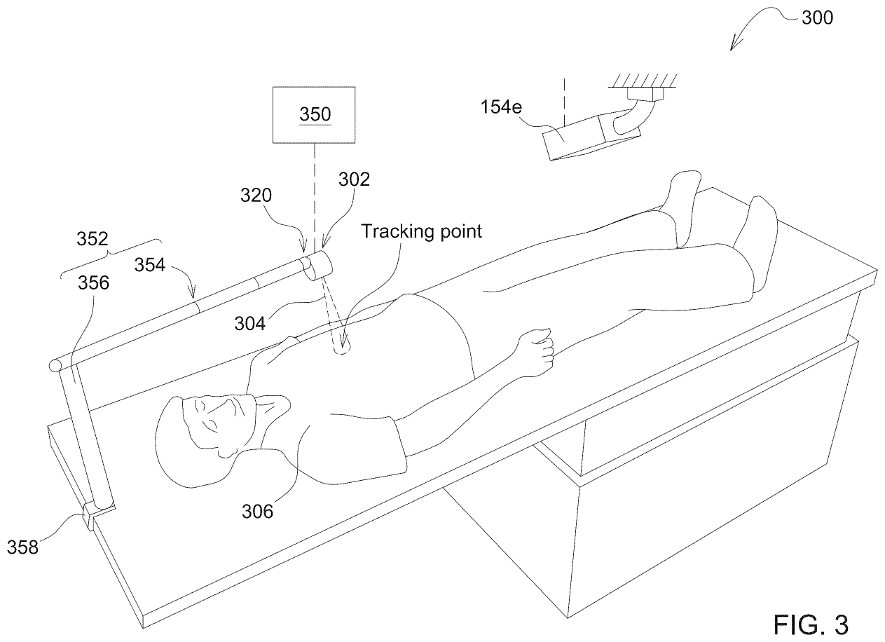

FIG. 3 illustrates a motion measuring apparatus according to at least one example embodiment. In the embodiment shown in FIG. 3, motion may be monitored using an event camera without a conventional frame-based camera, thus, reducing the analysis time and data and resulting in a faster response time.

The motion measuring apparatus 300 may be used with the radiation treatment system 10, or with other medical devices or systems for respiratory gating, respiration motion monitoring, heart monitoring and/or other motion from the patient. The apparatus 300 includes an energy device 302 configured to emit energy 304 towards a torso of a patient 306; and a processing unit 350 coupled to the energy device 302, wherein the processing unit 350 is configured to receive event data from the event camera 154e, and determine a breathing characteristic of the patient 306. It should be noted that the processing unit 350 is not limited to being physically coupled to the energy device 302, and that the processing unit 350 may be communicatively coupled to the energy device 302 (e.g., wirelessly). Also, in some cases, the processing unit 350 and the energy device 302 may be in different respective rooms. In other cases, they may be in the same room. While the energy device 302 and the event camera 154e are illustrated as separate, it should be understood that the event camera 154e and the energy device 302 may be integrated such as described above. Moreover, in other example embodiments, patient motion may be monitored passively without the energy device 302.

The energy device 302 is configured to provide an energy beam towards the patient 306, and the event camera 154e detects the motion of the energy beam on the patient. In some example embodiments, the energy device 302 is a light source shown in FIG. 1. By means of non-limiting examples, the energy device 302 may comprise a laser device configured to provide laser and/or to sense reflected laser or a light device configured to emit ultraviolet light or visible light.

The event camera 154e is configured to determine, or to provide information for determining, a distance from a fixed object. In particular, the motion measuring apparatus 300 may measure a distance relative to a fixed object, e.g., a surface of the patient support.

In other example embodiments, patient motion may be monitored passively without the energy device 302. Additionally or alternatively, an event camera 154e may be mounted on a support 354 instead of the energy device 302.

Returning to FIG. 3, in some cases, the processing unit 350 may be configured to determine the breathing characteristic of the patient 306 from the event data generated by the event camera 154e. By means of non-limiting examples, the breathing characteristic may be one or more breathing amplitude(s), one or more breathing phase(s), a period of a respiratory cycle, a breathing pattern, duration of breath holds or any combination of the foregoing.

In some cases, the processing unit 350 may include a breathing amplitude determination module configured to determine breathing amplitudes. In some cases, the distance information obtained by the processing unit 350 may be used as the breathing amplitudes. In other cases, the breathing amplitude determination module may perform scaling, averaging, normalization, or any combination of the foregoing, using the distance values to determine breathing amplitudes. The breathing amplitudes may be stored in a non-transitory medium for later use. In addition, or alternatively, the breathing amplitudes may be displayed on a screen for presentation to a user.

Also, in some cases, the processing unit 350 may include a breathing amplitude and phase determination module configured to determine breathing phases. In particular, by determining a plurality of positions of the patient's torso or fiducial over a period of time, the processing unit 350 can be configured to obtain a plurality of phase points and amplitudes that correspond to different levels of completeness of a breathing cycle at various time points. For example, a phase value may have a value from 0° to 360°, with 0° representing a beginning of a respiratory cycle, and 360° representing an end of the respiratory cycle.

In some embodiments, the determined phase and amplitude values may be used to gate an execution of a procedure, such as, to gate an application of a treatment radiation to the patient 306 for treatment, or to gate an application of an imaging radiation to the patient 306 for imaging purpose.

Returning to FIG. 3, in some embodiments, the processing unit 350 may be coupled to the energy device 302 using a cable. In other embodiments, the processing unit 350 may be coupled to the energy device 302 using a wireless system. In such cases, the energy device 302 may include a first wireless device, and the processing unit 350 may include a second wireless device for communication with the first wireless device.

As shown in the figure, the motion measuring apparatus 300 further includes a support structure 352, wherein the energy device 302 is moveably mounted to the support structure 352. In the illustrated embodiments, the support structure 352 includes a telescopic arm 354 and a vertical support 356. However, example embodiments are not limited thereto.

As shown in FIG. 3, the energy device 302 has an operative position that is above the torso of the patient 306. In other embodiments, the energy device 302 may have an operative position that is located at other places with respect to the patient 306. For example, the energy device 302 may be located above a head of the patient 306, or above the feet of the patient 306.

In addition, the motion measuring apparatus 300 further includes a mounting device 358 configured to mount the energy device 302 with respect to the patient support. In the illustrated example, the mounting device 358 is a clamp located at a bottom end of the vertical support 356 configured to detachably mount the vertical support 356 to the patient support. In other cases, the mounting device 358 may include a snap-fit connector, one or more screws, and/or other type of connectors for securing the motion measuring apparatus 300 relative to an object, such as the patient support, a gantry, a ceiling, a wall, etc.

Using event cameras for 3D surface tracking allows for low latency and high dynamic range measurement of the patient outline. The ability to perform low latency surface measurements at high spatial resolution allows the monitoring of patient motion for several time-critical applications such as treatment gating, collision detection, and system geometry verification. The high dynamic range of event cameras improves measurement accuracy on dark skin and eliminates problems with spotlights and overexposed parts of a scene. The expected high temporal resolution further allows the mounting on a moving gantry to prevent occlusion by the treatment machine.

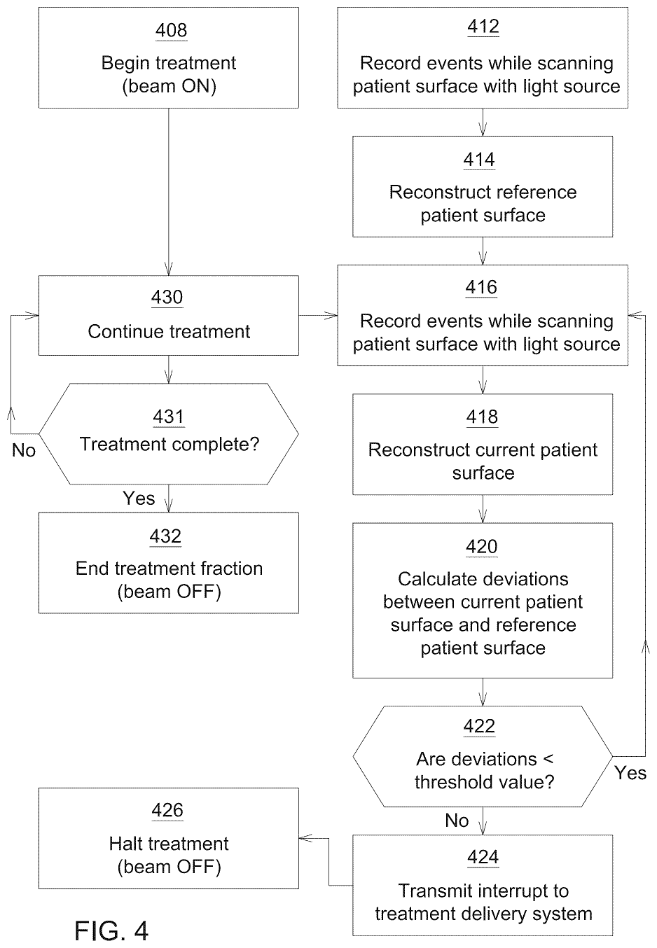

FIG. 4 illustrates a flowchart of a method 400 for a radiotherapy system, according to one or more embodiments. In some embodiments, the method 400 can be performed as part of a specific radiotherapy session or radiotherapy treatment fraction. The method 400 may include one or more operations, functions, or actions as illustrated by one or more of blocks 408-432. Although the blocks are illustrated in a sequential order, these blocks may be performed in parallel, and/or in a different order than those described herein. Also, the various blocks may be combined into fewer blocks, divided into additional blocks, and/or eliminated based upon the desired implementation. Although method 400 is described in conjunction with treatment delivery system 10, persons skilled in the art will understand that performance of method 400 by any suitably configured radiotherapy system with an event camera is within the scope of the present embodiments.

In step 408, in response to the one or more user inputs, radiation treatment system 10 begins treatment, and turns on a treatment beam. Additionally or alternatively, in some embodiments, as part of beginning treatment in step 408, the radiation treatment system 10 turns on an imaging beam, such as imaging X-rays, for acquiring images of a portion of patient anatomy that includes a target volume.

In step 412, in response to receiving a request from treatment delivery system 10, the control system 18 (or processing unit 156) acquires images or other image information of the patient surface using the at least one event camera 154e. Additionally or alternatively, in some embodiments, the control system 18 (or processing unit 156) acquires images or other image information of the patient surface in response to treatment beam 330 being turned on in step 408. For example, the treatment delivery system may also use the imaging X-rays. In step 414, the control system 18 (or processing unit 156) generates a reference patient surface based on the event date from the at least one event camera 154e and other image information (e.g., imaging X-rays) acquired in step 412. In some embodiments, the reference patient surface is then employed throughout a current treatment fraction for detecting excessive patient motion. Specifically, the reference patient surface is employed for a plurality of iterations of steps 416-422.

In step 416, the control system 18 (or processing unit 156) acquires event data of the patient surface using the at least one event camera 154e. In step 418, control system 18 (or processing unit 156) reconstructs a current patient surface based on the event data acquired in step 416.

In step 420, the control system 18 (or processing unit 156) calculates positional deviations between the current patient surface and the reference patient surface. In step 422, the control system 18 (or processing unit 156) determines whether the positional deviations determined in step 420 exceed allowable deviation thresholds. If yes, method 400 returns to step 416 and control system 18 (or processing unit 156) continues to monitor the patient surface for motion; if no, method 400 proceeds to step 424.

In step 424, the control system 18 (or processing unit 156) transmits an interrupt signal to treatment delivery system 200. Thus, the control system 18 (or processing unit 156) transmits the interrupt signal in response to a deviation of at least a portion of the current patient surface from the reference patient surface. In step 426, the treatment delivery system 10 halts delivery of the treatment fractions and turns off the treatment beam and/or imaging X-rays. In step 430, the control system 18 continues treatment. In some embodiments, continuation of treatment in step 430 includes delivery of the treatment beam, for example continuously or in pulses. In addition, in some embodiments, continuation of treatment in step 430 includes delivery of one or more imaging beams, such as imaging X-rays. In step 432, the control system 18 (or processing unit 156) determines whether the current treatment fraction has been completed. If yes, method 400 proceeds to step 432, where the control system 18 turns off the treatment beam and/or imaging X-rays and the treatment fraction ends normally; if no, the control system 18 returns to step 430 and the treatment fraction continues.

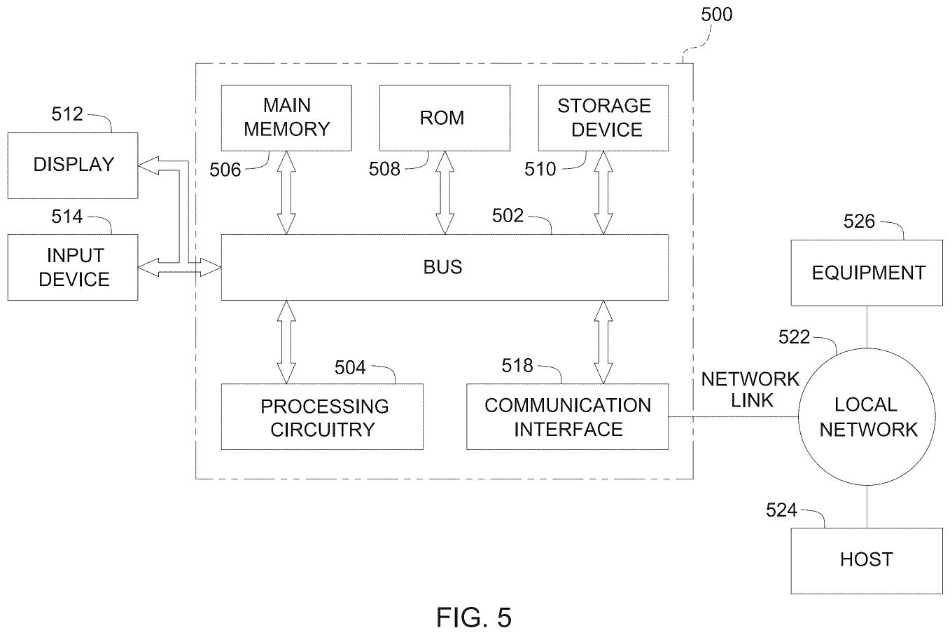

FIG. 5 is a block diagram illustrating an embodiment of a specialized control system 500 that can be used to implement various embodiments described herein. For example, the control system 500 may be configured to control the one or more event cameras in accordance with example embodiments.

Also, in some embodiments, the control system 500 may be used to implement the processing circuitry 54, the processing unit 156 or processing circuitry in the user interface device 90. The control system 500 may also be an example of any control system described herein.

The control system 500 includes a bus 502 or other communication mechanism for communicating information, and processing circuitry 504 (e.g., at least one processor and/or ASIC) coupled with the bus 502 for processing information. In examples where the processing circuitry 504 is hardware configured to executed stored instructions (e.g., a processor), the control system 500 also includes a main memory 506, such as a random-access memory (RAM) or other dynamic storage device, coupled to the bus 502 for storing information and instructions to be executed by the processing circuitry 504. The main memory 506 also may be used for storing temporary variables or other intermediate information during execution of instructions to be executed by the processing circuitry 504. The control system 500 further includes a read only memory (ROM) 508 or other static storage device coupled to the bus 502 for storing static information and instructions for the processing circuitry 504. A data storage device 510, such as a magnetic disk or optical disk, may be provided and coupled to the bus 502 for storing information and instructions.

The control system 500 may be coupled via the bus 502 to a display 512, such as a flat panel, for displaying information to a user. An input/output device 514, such as a touchscreen, is coupled to the bus 502 for communicating information and command selections to processing circuitry 504. Another type of user input device is cursor control 514, such as a mouse, a trackball, or cursor direction keys for communicating direction information and command selections to processing circuitry 504 and for controlling cursor movement on display 512. This input device typically has two degrees of freedom in two axes, a first axis (e.g., x) and a second axis (e.g., y), that allows the device to specify positions in a plane.

While the display 512 and I/O device 514 are shown outside of the control system 500, it should be understood that the display 512 and the I/O device 514 are part of the control system 500 such as shown in FIG. 5.

In some embodiments, the control system 500 can be used to perform various functions described herein. According to some embodiments, such use is provided by control system 500 in response to the processing circuitry 504 executing one or more sequences of one or more instructions contained in the main memory 506. Those skilled in the art will know how to prepare such instructions based on the functions, algorithms and methods described herein. Such instructions may be read into the main memory 506 from another processor-readable medium, such as storage device 510. Execution of the sequences of instructions contained in the main memory 506 causes the processing circuitry 504 to perform the process steps described herein. One or more processors in a multi-processing arrangement may also be employed to execute the sequences of instructions contained in the main memory 506. In alternative embodiments, hard-wired circuitry may be used in place of or in combination with software instructions to implement the various embodiments described herein. Thus, embodiments are not limited to any specific combination of hardware circuitry and software.

Transmission media includes coaxial cables, copper wire and fiber optics, including the wires that comprise the bus 502. Transmission media can also take the form of acoustic or light waves, such as those generated during radio wave and infrared data communications.

Various forms of processor-readable media may be involved in carrying one or more sequences of one or more instructions to the processing circuitry 504 for execution. For example, the instructions may initially be carried on a magnetic disk of a remote computer. The remote computer can load the instructions into its dynamic memory and send the instructions over a network, such as the Internet or a local network. A receiving unit local to the control system 500 can receive the data from the network and provide the data on the bus 502. The bus 502 carries the data to the main memory 506, from which the processing circuitry 504 retrieves and executes the instructions. The instructions received by the main memory 506 may optionally be stored on the storage device 510 either before or after execution by the processing circuitry 504.

The control system 500 also includes a communication interface 518 coupled to the bus 502. The communication interface 518 provides a two-way data communication coupling to a network link 520 that is connected to a local network 522. For example, the communication interface 518 may be an integrated services digital network (ISDN) card or a modem to provide a data communication connection to a corresponding type of telephone line. As another example, the communication interface 518 may be a local area network (LAN) card to provide a data communication connection to a compatible LAN. Wireless links may also be implemented. In any such implementation, the communication interface 518 sends and receives electrical, electromagnetic or optical signals that carry data streams representing various types of information.

The network link 520 typically provides data communication through one or more networks to other devices. For example, the network link 520 may provide a connection through local network 522 to a host computer 524 or to equipment 526 such as a radiation beam source or a switch operatively coupled to a radiation beam source. The data streams transported over the network link 520 can comprise electrical, electromagnetic or optical signals. The signals through the various networks and the signals on the network link 520 and through the communication interface 518, which carry data to and from the control system 500, are exemplary forms of carrier waves transporting the information. The control system 500 can send messages and receive data, including program code, through the network(s), the network link 520, and the communication interface 518.

Although the terms first, second, etc. may be used herein to describe various elements, these elements should not be limited by these terms. These terms are only used to distinguish one element from another. For example, a first element could be termed a second element, and similarly, a second element could be termed a first element, without departing from the scope of this disclosure. As used herein, the term “and/or,” includes any and all combinations of one or more of the associated listed items.

When an element is referred to as being “connected,” or “coupled,” to another element, it can be directly connected or coupled to the other element or intervening elements may be present. By contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Specific details are provided in the following description to provide a thorough understanding of example embodiments. However, it will be understood by one of ordinary skill in the art that example embodiments may be practiced without these specific details. For example, systems may be shown in block diagrams so as not to obscure the example embodiments in unnecessary detail. In other instances, well-known processes, structures and techniques may be shown without unnecessary detail in order to avoid obscuring example embodiments.

As discussed herein, illustrative embodiments will be described with reference to acts and symbolic representations of operations (e.g., in the form of flow charts, flow diagrams, data flow diagrams, structure diagrams, block diagrams, etc.) that may be implemented as program modules or functional processes include routines, programs, objects, components, data structures, etc., that perform particular tasks or implement particular abstract data types and may be implemented using existing hardware, for example, processing or control circuitry such as, but not limited to, one or more processors, one or more Graphics Processing Units (GPUs), one or more Central Processing Units (CPUs), one or more controllers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUs), one or more microprocessors, one or more Application Specific Integrated Circuits (ASICs), or any other device or devices capable of responding to and executing instructions in a defined manner.

Although a flow chart may describe the operations as a sequential process, many of the operations may be performed in parallel, concurrently or simultaneously. In addition, the order of the operations may be re-arranged. A process may be terminated when its operations are completed, but may also have additional steps not included in the figure. A process may correspond to a method, function, procedure, subroutine, subprogram, etc. When a process corresponds to a function, its termination may correspond to a return of the function to the calling function or the main function.

As disclosed herein, the term “memory,” “storage medium,” “processor readable medium,” “computer readable storage medium” or “non-transitory computer readable storage medium” may represent one or more devices for storing data, including read only memory (ROM), random access memory (RAM), magnetic RAM, core memory, magnetic disk storage mediums, optical storage mediums, flash memory devices and/or other tangible machine-readable mediums for storing information. The term “computer-readable medium” may include, but is not limited to, portable or fixed storage devices, optical storage devices, and various other mediums capable of storing, containing or carrying instruction(s) and/or data.

Furthermore, example embodiments may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium such as a computer readable storage medium. When implemented in software, a processor or processors will perform the necessary tasks. For example, as mentioned above, according to one or more example embodiments, at least one memory may include or store computer program code, and the at least one memory and the computer program code may be configured to, with at least one processor, cause a network element or network device to perform the necessary tasks. Additionally, the processor, memory and example algorithms, encoded as computer program code, serve as means for providing or causing performance of operations discussed herein.

The terms “including” and/or “having,” as used herein, are defined as comprising (i.e., open language). The term “coupled,” as used herein, is defined as connected, although not necessarily directly, and not necessarily mechanically. Terminology derived from the word “indicating” (e.g., “indicates” and “indication”) is intended to encompass all the various techniques available for communicating or referencing the object/information being indicated. Some, but not all, examples of techniques available for communicating or referencing the object/information being indicated include the conveyance of the object/information being indicated, the conveyance of an identifier of the object/information being indicated, the conveyance of information used to generate the object/information being indicated, the conveyance of some part or portion of the object/information being indicated, the conveyance of some derivation of the object/information being indicated, and the conveyance of some symbol representing the object/information being indicated.

According to example embodiments, medical systems, may be (or include) hardware, firmware, hardware executing software or any combination thereof. Such hardware may include processing or control circuitry such as, but not limited to, one or more processors (CPUs), one or more GPUs, one or more controllers, one or more ALUs, one or more DSPs, one or more microcomputers, one or more FPGAs, one or more SoCs, one or more PLUs, one or more microprocessors, one or more ASICs, or any other device or devices capable of responding to and executing instructions in a defined manner.

Benefits, other advantages, and solutions to problems have been described above with regard to specific embodiments. However, the benefits, advantages, solutions to problems, and any element(s) that may cause or result in such benefits, advantages, or solutions, or cause such benefits, advantages, or solutions to become more pronounced are not to be construed as a critical, required, or essential feature or element of any or all the claims.

### Non-limiting Illustrative Embodiments

Illustrative embodiment 1 includes a system comprising a radiotherapy machine; at least one event camera; and processing circuitry configured to operate at least one of the radiotherapy machine or the at least one event camera.

Illustrative embodiment 2 includes the system of illustrative embodiment 1, wherein the at least one event camera is coupled to the radiotherapy machine.

Illustrative embodiment 3 includes the system of any one of illustrative embodiments 1-2, wherein the radiotherapy machine includes a gantry and the at least one event camera is on the gantry.

Illustrative embodiment 4 includes the system of any one of illustrative embodiments 1-3, wherein the at least one event camera is configured to capture event data associated with a patient, and the processing circuitry is configured to cause the system to obtain a measured surface of a patient based on event data of the at least one event camera.

Illustrative embodiment 5 includes the system of any one of illustrative embodiments 1-4, further comprising at least one light source, wherein the processing circuitry is configured to cause the system to, control the at least one light source to emit light onto the patient, and obtain the measured surface of the patient further based on the emitted light onto the patient.

Illustrative embodiment 6 includes the system of illustrative embodiment 5, wherein the at least one light source is a laser.

Illustrative embodiment 7 includes the system of illustrative embodiment 5, wherein the at least one light source is a structured light projector.

Illustrative embodiment 8 includes the system of any one of illustrative embodiments 1-7, further comprising at least one frame-based camera, wherein the at least one event camera includes a higher temporal resolution than the at least one frame-based camera.

Illustrative embodiment 9 includes the system of any one of illustrative embodiments 1-8, wherein the at least one event camera includes at least two event cameras.

Illustrative embodiment 10 includes the system of illustrative embodiment 9, further comprising a different laser associated with each event camera, wherein each event camera is configured to capture event data corresponding to light emitted from the associated event camera.

Illustrative embodiment 11 includes the system of any one of illustrative embodiments 1-10, wherein the at least one event camera is on a wall of a room including the radiotherapy machine.

Illustrative embodiment 12 includes the system of any one of illustrative embodiments 1-11, wherein the at least one event camera is on a ceiling of a room including the radiotherapy machine.

Illustrative embodiment 13 includes the system of illustrative embodiment 12, wherein the at least one event camera is mounted to a pod, the pod being mounted to the ceiling.

Illustrative embodiment 14 includes the system of illustrative embodiment 12, wherein the at least one event camera is mounted to a static structure, the static structure being mounted to the ceiling.

Illustrative embodiment 15 includes the system of any one of illustrative embodiments 1-14, further comprising a patient couch, wherein the at least one event camera is on the patient couch.

Illustrative embodiment 16 includes the system of any one of illustrative embodiments 1-15, further comprising a patient couch, the radiotherapy machine defining a bore to receive the patient couch, wherein the at least one event camera is on a surface defining the bore.

Illustrative embodiment 17 includes the system of any one of illustrative embodiments 1-16, wherein the at least one event camera is configured to capture event data associated with at least one of a patient or a component of the system, and the processing circuitry is configured to cause the system to detect a possible collision based on the captured event data.

Illustrative embodiment 18 includes the system of any one of illustrative embodiments 1-17, wherein the at least one event camera includes at least two event cameras separated by a distance, each of the at least two event cameras is configured to capture event data associated with motion of a patient, and the processing circuitry is configured to cause the system to obtain a measured surface of a patient based on the event data from the at least two event cameras and the distance.

Illustrative embodiment 19 includes the system of any one of illustrative embodiments 1-18, wherein the processing circuitry is configured to cause the system to obtain a reference patient surface; acquire at least one image of the patient using the at least one event camera; and control the radiotherapy machine based on the reference patient surface and the at least one acquired image.

Illustrative embodiment 20 includes the system of illustrative embodiment 19, wherein the processing circuitry is configured to cause the system to determine a deviation between the at least one acquired image and the reference patient surface, determine whether the deviation is less than a threshold value, and control the radiotherapy machine based on whether the deviation is less than the threshold value.

Illustrative embodiment 21 includes a computer-implemented method comprising obtaining event data from at least one event camera and controlling a radiotherapy machine based on the event data.

Illustrative embodiment 22 includes the method of illustrative embodiment 21 wherein the event data is associated with a patient and the method further comprises obtaining a measured surface of the patient based on the event data.

Illustrative embodiment 23 includes the method of any one of illustrative embodiments 21-22 further comprising controlling at least one light source to emit light onto a patient and obtain a measured surface of the patient based on the emitted light onto the patient.

Illustrative embodiment 24 includes the method of illustrative embodiment 23, wherein the at least one light source is a structured light projector.

Illustrative embodiment 25 includes the method of any one of illustrative embodiments 21-24, further comprising obtaining image data from at least one frame-based camera, the at least one event camera includes a higher temporal resolution than the at least one frame-based camera, and controlling the radiotherapy machine further based on the image data.

Illustrative embodiment 26 includes the method of any one of illustrative embodiments 21-25, wherein the obtaining obtains the event data from at least two event cameras.

Illustrative embodiment 27 includes the method of illustrative embodiment 26, wherein each event camera is associated with a different laser, and the method further comprises, for each event camera, capturing event data corresponding to light emitted from the associated event camera.

Illustrative embodiment 28 includes the method of any one of illustrative embodiments 21-28 wherein the at least one event camera is on a wall of a room including the radiotherapy machine.

Illustrative embodiment 29 includes the method of any one of illustrative embodiments 21029, wherein the at least one event camera is on a ceiling of a room including the radiotherapy machine.

Illustrative embodiment 30 includes the method of illustrative embodiment 29, wherein the at least one event camera is mounted to a pod, the pod being mounted to the ceiling.

Illustrative embodiment 31 includes the method of illustrative embodiment 29, wherein the at least one event camera is mounted to a static structure, the static structure being mounted to the ceiling.

Illustrative embodiment 32 includes the method of any one of illustrative embodiments 21-31, wherein the at least one event camera is on a patient couch.

Illustrative embodiment 33 includes the method of any one of illustrative embodiments 21-32, wherein the at least one event camera is on a surface defining a bore to receive a patient couch.

Illustrative embodiment 34 includes the method of any one of illustrative embodiments 21-33, wherein the obtaining obtains event data associated with at least one of a patient or a component of the system, and the method further comprises detecting a possible collision based on the event data associated with at least one of the patient or the component of the system.

Illustrative embodiment 35 includes the method of any one of illustrative embodiments 21-34, wherein the at least one event camera includes at least two event cameras separated by a distance, the obtaining obtains event data associated with motion of a patient, and the method further comprises obtaining a measured surface of a patient based on the event data from the at least two event cameras and the distance.

Illustrative embodiment 36 includes the method of any one of illustrative embodiments 21-35, further comprising obtaining a reference patient surface; acquiring at least one image of the patient using the at least one event camera; and controlling the radiotherapy machine based on the reference patient surface and the at least one acquired image.

Illustrative embodiment 37 includes the method of illustrative embodiment 36, further comprising determining a deviation between the at least one acquired image and the reference patient surface, determining whether the deviation is less than a threshold value, and controlling the radiotherapy machine based on whether the deviation is less than the threshold value.

Illustrative embodiment 38 includes a computer-readable medium having instructions, when executed by processing circuitry of a system, causes the system to perform the method of any one of illustrative embodiments 21-37.

Illustrative embodiment 39 includes a computer program having instructions, when executed by processing circuitry of a system, causes the system to perform the method of any one of illustrative embodiments 21-37.

What is claimed is

1. A system comprising:
a radiotherapy machine;
at least one event camera; and
processing circuitry configured to operate at least one of the radiotherapy machine or the at least one event camera.

2. The system of claim 1, wherein the at least one event camera is coupled to the radiotherapy machine.

3. The system of claim 2, wherein the radiotherapy machine includes a gantry and the at least one event camera is on the gantry.

4. The system of claim 1, wherein
the at least one event camera is configured to capture event data associated with a patient, and
the processing circuitry is configured to cause the system to obtain a measured surface of a patient based on event data of the at least one event camera.

5. The system of claim 4, further comprising:
at least one light source, wherein the processing circuitry is configured to cause the system to,
control the at least one light source to emit light onto the patient, and
obtain the measured surface of the patient further based on the emitted light onto the patient.

6. The system of claim 5, wherein the at least one light source is a laser.

7. The system of claim 5, wherein the at least one light source is a structured light projector.

8. The system of claim 1, further comprising:
at least one frame-based camera, wherein the at least one event camera includes a higher temporal resolution than the at least one frame-based camera.

9. The system of claim 1, wherein the at least one event camera includes at least two event cameras.

10. The system of claim 9, further comprising:
a different laser associated with each event camera, wherein each event camera is configured to capture event data corresponding to light emitted from the associated event camera.

11. The system of claim 1, wherein the at least one event camera is on a wall of a room including the radiotherapy machine.

12. The system of claim 1, wherein the at least one event camera is on a ceiling of a room including the radiotherapy machine.

13. The system of claim 12, wherein the at least one event camera is mounted to a pod, the pod being mounted to the ceiling.

14. The system of claim 12, wherein the at least one event camera is mounted to a static structure, the static structure being mounted to the ceiling.

15. The system of claim 1, further comprising:
a patient couch, wherein the at least one event camera is on the patient couch.

16. The system of claim 1, further comprising:
a patient couch, the radiotherapy machine defining a bore to receive the patient couch, wherein the at least one event camera is on a surface defining the bore.

17. The system of claim 1, wherein
the at least one event camera is configured to capture event data associated with at least one of a patient or a component of the system, and
the processing circuitry is configured to cause the system to detect a possible collision based on the captured event data.

18. The system of claim 1, wherein
the at least one event camera includes at least two event cameras separated by a distance, each of the at least two event cameras is configured to capture event data associated with motion of a patient, and
the processing circuitry is configured to cause the system to obtain a measured surface of a patient based on the event data from the at least two event cameras and the distance.

19. The system of claim 1, wherein the processing circuitry is configured to cause the system to,
obtain a reference patient surface;
acquire at least one image of the patient using the at least one event camera; and
control the radiotherapy machine based on the reference patient surface and the at least one acquired image.

20. The system of claim 19, wherein the processing circuitry is configured to cause the system to,
determine a deviation between the at least one acquired image and the reference patient surface,
determine whether the deviation is less than a threshold value, and
control the radiotherapy machine based on whether the deviation is less than the threshold value.
